(19) United States
(12) Patent Application Publication
(10) Pub. No.: US 2022/0142579 A1
(43) Pub. Date: May 12, 2022

(54) BIOLOGICAL DATA ACQUISITION APPARATUS AND BIOLOGICAL DATA PROCESSING APPARATUS USING THE SAME

(71) Applicant: UIF (University Industry Foundation), Yonsei University, Seoul (KR)

(72) Inventors: Wonhyoung RYU, Goyang-si (KR); Jiyong LEE, Seoul (KR); Jeong Min KIM, Seoul (KR)

(73) Assignee: UIF (University Industry Foundation), Yonsei University, Seoul (KR)

(21) Appl. No.: 17/520,130

(22) Filed: Nov. 5, 2021

(30) Foreign Application Priority Data
Nov. 6, 2020 (KR) 10-2020-0148092

(51) Int. Cl. A61B 5/00; A61B 5/1486

(52) U.S. Cl. CPC A61B 5/6853; A61M 2025/1086; A61B 2562/04; A61B 5/14865

(57) ABSTRACT

Provided are an apparatus for acquiring biological data and an apparatus for processing biological data. The apparatus for being inserted into a living body and acquiring biological information includes a balloon inserted into the living body to expand or contract, one or more electrodes disposed on a surface of the balloon, and one or more fine protrusions disposed on surfaces of the electrodes to come into contact with the living body.

### CROSS REFERENCE TO RELATED APPLICATION

This application claims priority to Korean Patent Application No. 10-2020-0148092 (filed on Nov. 6, 2020), which is hereby incorporated by reference in its entirety.

### BACKGROUND

The present invention relates to an apparatus for acquiring biological data and an apparatus for processing biological data using the same.

To visually observe the inside of the human body with a biological information acquisition apparatus according to the related art, a chemical, such as a contrast agent, is injected into the human body and is observed externally, or an ultrasound wave and the like are emitted into the human body and are observed.

### SUMMARY

To visually identify a lesion and the like, magnetic resonance imaging (MRI) equipment, computed tomography (CT) equipment, ultrasound equipment, etc. according to the related art are used. However, images of the related art have low resolution, and in particular, it is not possible to know where a lesion is locally located, material property information of the corresponding location, etc.

The present invention is directed to providing a technology for acquiring biological information using a minimally invasive method or a non-invasive method and processing the biological information.

According to an aspect of the present invention, there is provided an apparatus for being inserted into a living body and acquiring biological information, the apparatus including a balloon inserted into the living body to expand or contract and one or more electrodes disposed on a surface of the balloon.

According to another aspect of the present invention, there is provided an apparatus for processing biological information, the apparatus including a biological information acquisition part including a balloon inserted into a living body to expand or contract and one or more electrodes disposed on a surface of the balloon and a processing unit configured to process biological information acquired by the biological information acquisition part.

The apparatus for acquiring biological information may include one or more fine protrusions disposed on surfaces of the electrodes to come into contact with the living body.

The apparatus for acquiring biological information may be inserted into any one or more of a blood vessel and a body cavity of the living body to acquire the biological information.

The electrodes may include a working electrode and a counter electrode, and the working electrode and the counter electrode may detect any one or more of a current and a voltage generated by oxidation and reduction reactions which occur in the living body coming into contact with the apparatus.

The fine protrusions disposed on the working electrode and the counter electrode may invade the living body such that the oxidation and reduction reactions occur.

The fine protrusions may include a material which provides an electrical signal corresponding to deformation caused by an external force.

The fine protrusions may be coated with a flexible material.

The fine protrusions may include a mixture of a flexible material and the material which provides an electrical signal corresponding to deformation caused by an external force.

The fine protrusions may have a form of a pyramid or a cone.

The electrodes may include a first electrode and a second electrode to which an electrical signal is applied and which detect an electrical signal obtained by providing the electrical signal to the living body, and the living body and the fine protrusions formed on the first electrode and the second electrode may form a path for the electrical signals.

The electrical signal applied to the first electrode may be any one of a voltage signal and a current signal, and the electrical signal detected by the second electrode may be any one of the voltage signal and the current signal.

The fine protrusions may have any one form of a cylinder, a prism, a truncated cone, and a prismatoid.

The fine protrusions may include a conductive material.

The fine protrusions may include a swelling material which absorbs a biological substance at a location where the apparatus is present.

The electrodes may include a swelling material which absorbs a biological substance at a location where the apparatus is present.

The electrodes may be disposed in one or more pairs spaced apart from each other on a surface circumference of the balloon.

The apparatus may further include a guide tube through which a fluid flows in or out to expand or contract the balloon and a wire connected to the electrodes.

According to the present embodiment, the apparatus for acquiring biological information can be inserted into a living body and acquire biological information in a minimally invasive manner or non-invasive manner. Also, it is possible to easily acquire property information of a living body that cannot be obtained in the related art.

### BRIEF DESCRIPTION OF THE DRAWINGS

FIG. 1 is a perspective view of an apparatus for acquiring biological information according to an exemplary embodiment;

FIG. 2 is a diagram illustrating an overview of an apparatus for processing biological information including an apparatus for acquiring biological information inserted into the human body and a processing unit for processing biological information acquired by the apparatus for acquiring biological information;

FIG. 3 is a schematic cross-sectional view illustrating the apparatus for acquiring biological information according to the exemplary embodiment that is inserted into and operating in a blood vessel;

FIGS. 4A and 4B are graphs illustrating an impedance change measured with a changing frequency when a lipid and the like are not accumulated and an impedance change measured with a changing frequency when a lipid and the like are accumulated, respectively;

FIGS. 5A and 5B are schematic cross-sectional views illustrating an electrochemical analysis performed by the apparatus for acquiring biological information according to the exemplary embodiment;

FIGS. 6A and 6B are a diagram illustrating an example of the apparatus for acquiring biological information according to the exemplary embodiment that is inserted into the aorta, which is a cardiovascular blood vessel, and detects the rigidity of the aorta and a diagram of an electrode and a fine protrusion, respectively;

FIGS. 7A and 7B are a diagram illustrating an example of the apparatus for acquiring biological information according to the exemplary embodiment that is inserted into the aorta, which is a cardiovascular blood vessel, and detects the rigidity of the aorta in which fine protrusions come into contact with the inner wall of the aorta and are deformed and a diagram illustrating a detailed structure of a fine protrusion, respectively;

FIG. 8 is a diagram schematically illustrating an operation in which fine protrusions formed of a swelling material according to the exemplary embodiment may absorb biological substances of contacting or invaded tissue to expand and then extract the biological substances to the outside of a living body according to another exemplary embodiment;

FIGS. 9A to 9C are an example of simulating a blood vessel with arteriosclerosis, an example of the path of a current when an electrical signal is provided using an apparatus (10) for acquiring biological information according to the exemplary embodiment, and a set of graphs illustrating impedance detection results of the above experiment, respectively; and

FIGS. 10A and 10B are a set of views of a blood vessel phantom, which is obtained by freezing and thawing 10% polyvinyl alcohol (PVA) six times to increase stiffness and putting 200 μm of animal fat on the inner wall, and the aorta of a pig and a graph illustrating mechanical property values measured with the apparatus (10) for acquiring biological information according to the exemplary embodiment when a fat component is in a blood vessel and is not in a blood vessel, respectively.

### DETAILED DESCRIPTION

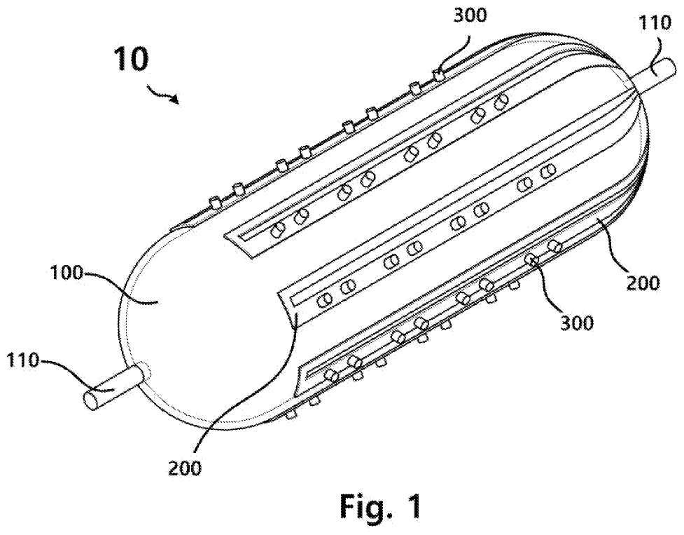

Hereinafter, exemplary embodiments of the present invention will be described in detail with reference to the accompanying drawings. FIG. 1 is a perspective view of an apparatus 10 for acquiring biological information according to an exemplary embodiment. Referring to FIG. 1, the apparatus 10 is inserted into a living body to acquire biological information and includes a balloon 100 that may be inserted into the living body and may expand or contract, two or more electrodes 200 that are disposed on the surface of the balloon 100, one or more fine protrusions 300 disposed on the surfaces of the electrodes 200 to come into contact with the living body.

According to the exemplary embodiment, the apparatus 10 may be inserted into a body cavity of the living body, such as the vascular system including brain blood vessels and heart blood vessels, a cavity of the digestive system including the esophagus, the stomach, the large intestine, the small intestine, the biliary tract, and the liver, a cavity of the respiratory system including the nose, the mouth, the ears, and the airway, and a cavity of the excretive system including the urethra and the anus, to acquire biological information.

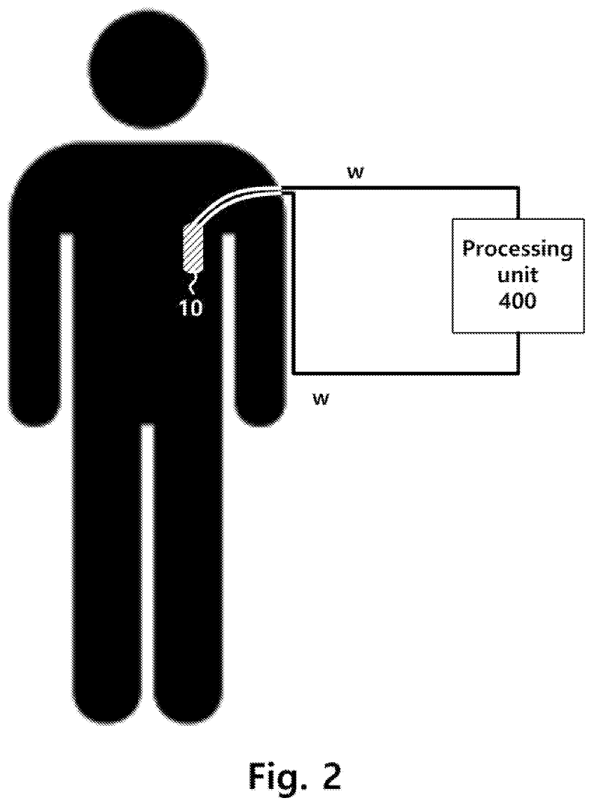

FIG. 2 is a diagram illustrating an overview of an apparatus for processing biological information including the apparatus 10 for acquiring biological information inserted into the human body and a processing unit 400 for processing biological information acquired by the apparatus 10 for acquiring biological information. Referring to FIGS. 1 and 2, the apparatus for processing biological information includes the apparatus 10 for acquiring biological information including the balloon 100 inserted into a living body to acquire biological information, the two or more electrodes 200 disposed on the surface of the balloon 100, and the one or more fine protrusions 300 disposed on the surfaces of the electrodes 200 to come into contact with the living body and a processing unit 400 for processing biological information acquired by the apparatus 10 for acquiring biological information.

Referring to FIGS. 1 and 2, the apparatus 10 for acquiring biological information includes the balloon 100 inserted into a blood vessel or a cavity of a living body. According to the exemplary embodiment, the balloon 100 may have different sizes depending on the size of a target into which the balloon 100 is inserted. As an example, the balloon 100 inserted into a blood vessel, such as a brain blood vessel, may have a length of 1 cm to 5 cm and a diameter of 2 mm to 4 mm. As another example, the balloon 100 inserted into the esophagus may have a diameter of 1 cm to 3 cm and may have different lengths depending on a location into which the balloon 100 is inserted as described above. In other words, the diameter of the balloon 100 may have any value of 1 mm to 30 mm and may vary depending on a target from which information will be acquired.

When a fluid, such as a gas or liquid, flows into the balloon 100 through a guide tube 110, the balloon 100 may expand. Also, when the fluid flows out, the balloon 100 may contract. The balloon 100 does not burst in the living body during expansion or contraction and may be formed of a material which does not react with body fluids, such as blood and gastric juices, in the living body.

The one or more electrodes 200 may be disposed on the surface of the balloon 100. The electrodes 200 may be disposed not to be open-circuited or short-circuited during expansion and contraction of the balloon 100. According to the exemplary embodiment, the electrodes 200 may be formed by sputtering, deposition, etc. on the balloon 100. The electrodes 200 may be formed of a conductive material which does not react with body fluids such as gastric juices and blood. According to the exemplary embodiment, the electrodes 200 are connected to a wire w disposed in the guide tube 110, and the wire w may extend to the outside of the living body and be connected to the processing unit 400.

The one or more fine protrusions 300 may be disposed on the surfaces of the electrodes 200. The fine protrusions 300 are elements that directly come into contact with biological tissue and may vary in shape. According to the exemplary embodiment, when the fine protrusions 300 are required to invade biological tissue or to be easily deformed by an external force, the fine protrusions 300 may be formed in a pyramid or cone shape having a sharp tip.

According to another exemplary embodiment, when the fine protrusions 300 are required not to invade biological tissue but to come into contact with biological tissue, the fine protrusions 300 preferably have the shape of any one of a cylinder, a prism, a truncated cone, and a prismatoid.

However, the shapes of the fine protrusions 300 are merely exemplary, and when the fine protrusions 300 are required to invade biological tissue or to be easily deformed by an external force, the fine protrusions 300 may also have the shape of a cylinder, a prism, a truncated cone, a prismatoid, etc. Also, when the fine protrusions 300 are not required to invade biological tissue, the fine protrusions 300 may also have the shape of a pyramid or cone.

A material of the fine protrusions 300 may vary depending on biological information to be acquired. According to the exemplary embodiment, when the fine protrusions 300 are required to provide an electrical signal to biological tissue and receive an electrical signal from the biological tissue, the fine protrusions 300 preferably include a conductive material.

Also, when the fine protrusions 300 are required to output an electrical signal corresponding to the rigidity of biological tissue, the fine protrusions 300 preferably include a material that outputs an electrical signal according to the degree of deformation of the fine protrusions 300. As an example, the fine protrusions 300 preferably include piezoelectric ceramic particles, such as barium titanate, or a piezoelectric material, such as polyvinylidene fluoride (PVDF).

The processing unit 400 is connected to the apparatus 10 for acquiring biological information and processes an electrical signal provided by the apparatus 10 for acquiring biological information. According to the exemplary embodiment, the processing unit 400 is connected to the wire w connected to the electrodes 200 and detects and processes an electrical signal and the like acquired by the apparatus 10 for acquiring biological information.

For example, the processing unit 400 may run a biological information computation program through a computation device (not shown) and perform impedance calculation, living body rigidity calculation, and substance detection in the body based on an electrochemical reaction from the detected electrical signal. Also, the processing unit 400 may introduce or discharge a fluid through the guide tube 110 to expand or contract the balloon 100 to a desired pressure.

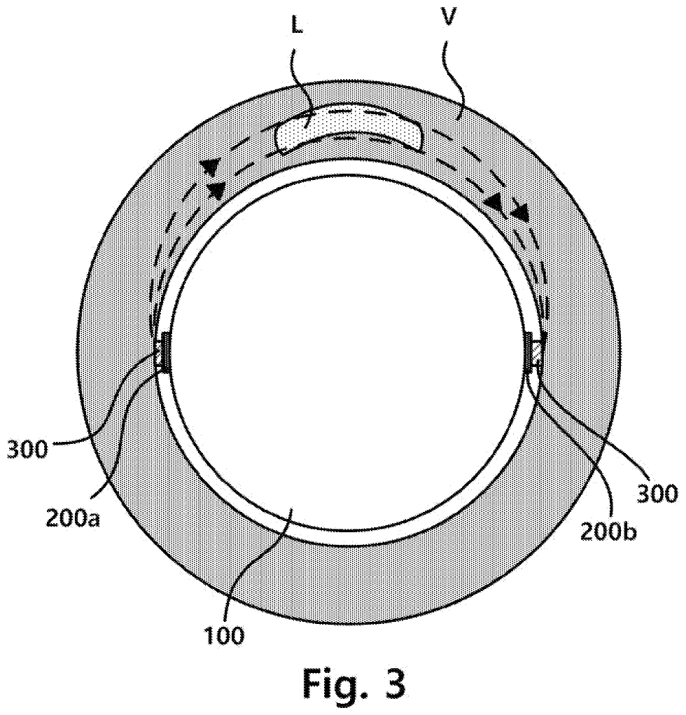

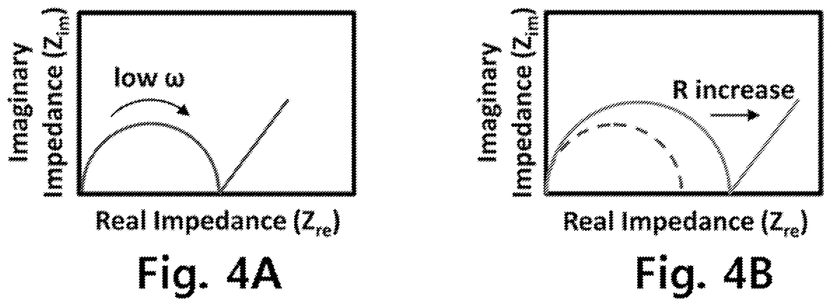

FIG. 3 is a schematic cross-sectional view illustrating the apparatus 10 for acquiring biological information according to the exemplary embodiment that is inserted into a blood vessel and operating, and FIGS. 4A and 4B are graphs illustrating an impedance change measured with a changing frequency when a lipid and the like are not accumulated and an impedance change measured with a changing frequency when a lipid and the like are accumulated, respectively. Referring to FIG. 3, the apparatus 10 for acquiring biological information is inserted into, for example, a blood vessel V and expanded by a fluid provided through the guide tube 110 such that fine protrusions 300 disposed on a first electrode 200a and fine protrusions 300 disposed on a second electrode 200b come into contact with the inner wall of the blood vessel V.

The processing unit 400 (see FIG. 2) applies an electrical signal to the first electrode 200a and the second electrode 200b connected through the wire w (see FIG. 2). The blood vessel V in contact with the fine protrusions 300 forms a conductive path through which an electrical signal flows.

Referring to FIGS. 4A and 4B, an impedance change of a conductive path is measured while the frequency of an electrical signal provided through the fine protrusions 300 is changed. When vulnerable plague L is formed because a lipid and the like are accumulated in a blood vessel or a blood vessel is calcified, the real number component and the imaginary number component of impedance are changed more greatly than a case in which the vulnerable plague L is not formed. The amount of change corresponds to the size of the vulnerable plague L.

An alternating current (AC) voltage is applied through the fine protrusions 300 disposed on the first and second electrodes 200, and the corresponding AC signal is measured, or an AC current is applied through the fine protrusions 300, and the corresponding AC voltage signal is measured. In this way, it is possible to determine whether the vulnerable plague L is formed in the blood vessel V and the size of the vulnerable plague L.

In vascular images acquired by existing image analysis devices, blood vessels are shown in black, and only the narrowing of a width of the blood vessel is confirmable. Even when magnetic resonance imaging (MRI) equipment, optical coherence tomography (OCT) equipment, or ultrasound equipment is used for a check, precise analysis is difficult. However, according to the exemplary embodiment, it is possible to know a local lipid distribution in a blood vessel, the degree of calcification, and the degree of vulnerable plague formation with higher accuracy and sensitivity than the related art. Further, the exemplary embodiment can be used in diagnosing a blood vessel at risk of rupture due to arteriosclerosis and the like in advance and preventing an unexpected event.

According to the exemplary embodiment, the plurality of fine protrusions 300 may be formed on the first electrode 200a and the second electrode 200b. In this case, a plurality of conductive paths may be formed between the plurality of fine protrusions 300 disposed on the first electrode 200a and the plurality of fine protrusions 300 disposed on the second electrode 200b. In this way, it is possible to precisely detect lipids, calcification, and the vulnerable plague L in the conductive paths.

Also, FIG. 3 illustrates that the first electrode 200a and the second electrode 200b form one pair of electrodes. However, according to an embodiment not shown in the drawings, two or more pairs of electrodes may be disposed to measure the impedance of a conductive path.

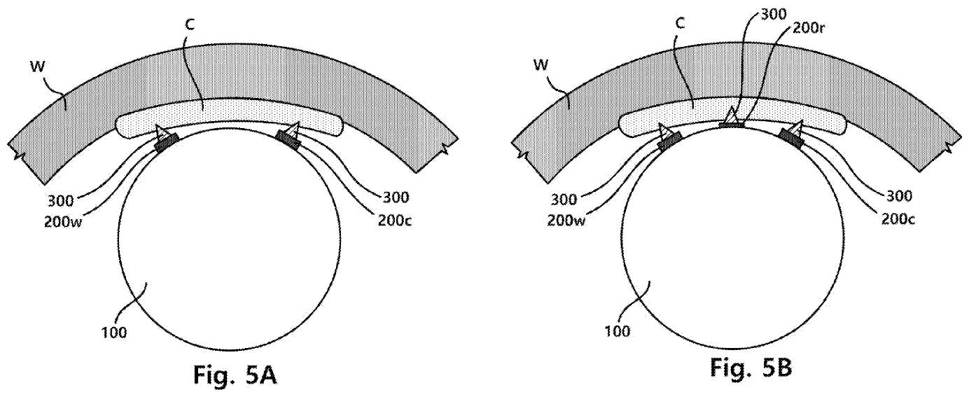

FIGS. 5A and 5B are schematic cross-sectional views illustrating an electrochemical analysis performed by the apparatus for acquiring biological information according to the exemplary embodiment. In the exemplary embodiment shown in FIGS. 5A and 5B, the apparatus 10 for acquiring biological information is inserted into a cavity of a living body. Referring to FIG. 5A, the apparatus 10 for acquiring biological information may include two electrodes. One of the electrodes may function as a working electrode 200w, and the other may function as a counter electrode 200c.

In the exemplary embodiment illustrated in FIG. 5B, the apparatus 10 for acquiring biological information may include three electrodes, which may function as a working electrode 200w, a counter electrode 200c, and a reference electrode 200r.

In the exemplary embodiments illustrated in FIGS. 5A and 5B, the apparatus 10 for acquiring biological information is inserted into a living body and disposed to come into contact with a target region C. The fine protrusions 300 may come into contact with the target region C or invade and come into contact with the target region C as shown in the exemplary embodiments shown in the drawing.

In the exemplary embodiment illustrated in FIG. 5A, between the working electrode 200w and the counter electrode 200c, a potential difference is caused by electrochemical reactions, such as oxidation and reduction reactions, and a current flows due to the reactions. The processing unit 400 may detect the potential difference and the current through the wire w.

Referring to FIG. 5B, between the working electrode 200w and the counter electrode 200c, a potential difference is caused by electrochemical reactions, such as oxidation and reduction reactions, and a current flows due to the reactions. However, a voltage drop IRdrop may be caused by the electrical resistance of the target region C such that an error may occur. When the resistance value of the target region C is large or a high current flows, the reference electrode 200r may be used for precise measuring.

When the three electrodes are used for measuring, a current flows between the working electrode 200w and the counter electrode 200c, and the electrical potential of the working electrode 200w is adjusted and measured on the basis of the reference electrode 200r. The potential difference between the working electrode 200w and the reference electrode 200r may be accurately measured regardless of the value of a current flowing due to electrochemical reactions.

As another example, when the working electrode 200w and the counter electrode 200c are included, the counter electrode 200c may be used as a pseudo reference electrode for precise measuring.

In the exemplary embodiments shown in the drawing, the processing unit 400 causes electrochemical reactions, such as oxidation and reduction reactions, at the working electrode 200w, scans a potential difference caused by the electrochemical reactions through the wire w, and detects a current caused by the electrochemical reactions. For example, the processing unit 400 detects a change in the current at a specific electrical potential while scanning the potential difference from 0 V to 1 V in units of 10 mV.

When a specific material is present, the current is suddenly changed at a specific electrical potential. Accordingly, from a voltage at which the current is suddenly changed, the processing unit 400 may determine whether the corresponding material is in the target region C and the amount of the material.

According to the related art, a region of interest is visually detected through an endoscope, and tissue of the region of interest is removed and examined. In this process, the tissue of body cavity may be injured. However, according to the exemplary embodiment, electrochemical analysis can be performed using the apparatus 10 for acquiring biological information without making a wound in a region of interest. From this, it is possible to determine whether a lesion, such as lipid, a polyp, and cancer, is present in a target region and detect a target protein, biomarker, etc.

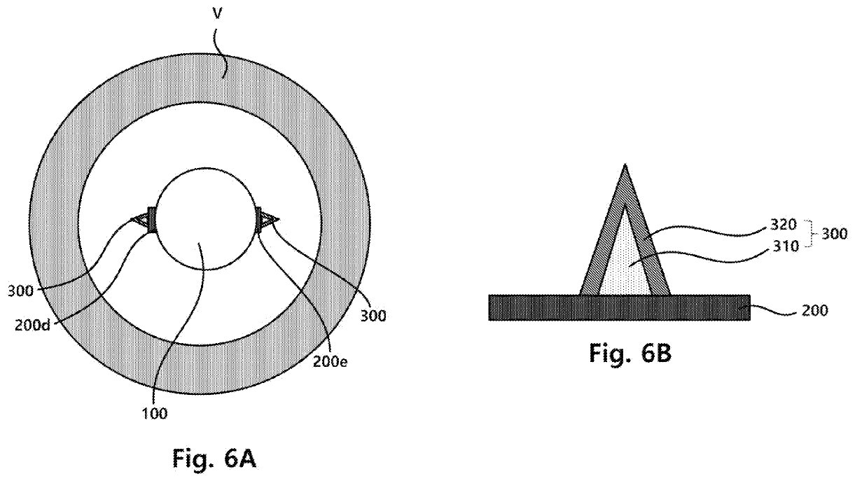

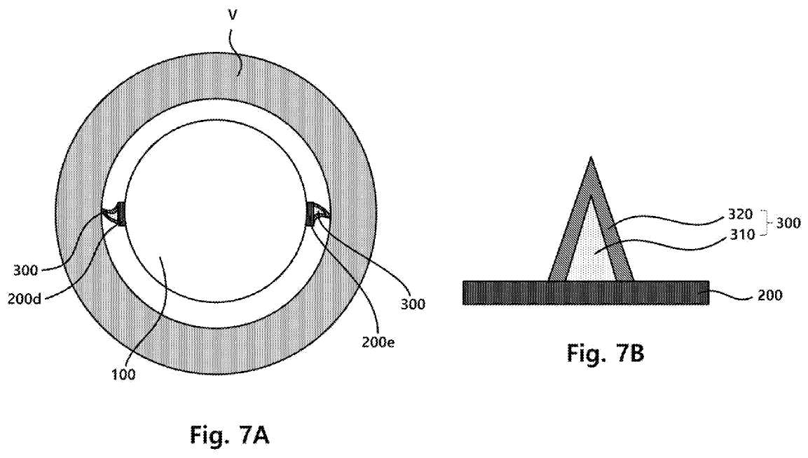

FIGS. 6 and 7 are schematic cross-sectional views illustrating an apparatus for acquiring biological information according to another exemplary embodiment. FIG. 6A is a diagram illustrating an example of the apparatus for acquiring biological information according to the exemplary embodiment that is inserted into the aorta V, which is a cardiovascular blood vessel, and detects the rigidity of the aorta, and FIG. 6B is a diagram illustrating an electrode 200 and a fine protrusion 300 in detail. Referring to FIG. 6A, a balloon 100 may be inserted into the artery V without being sufficiently inflated. Accordingly, a fine protrusion 300 disposed on a first electrode 200d and a fine protrusion 300 disposed on a second electrode 200e are not deformed.

Referring to FIG. 6B, the fine protrusion 300 may include an external force detector 310 and a flexible coating part 320. The fine protrusion 300 including the external force detector 310 and the flexible coating part 320 may be formed with a high aspect ratio to be easily deformed by an external force. The fine protrusion 300 is illustrated as a tetrahedron or a cone in the example but may be implemented in the form of a pillar, such as a cylinder, having a high height compared to the base area to be easily deformed by an external force.

According to the exemplary embodiment, the external force detector 310 may be formed of a flexible material to be easily deformed when an external force is provided and may also be formed of a material providing an electrical signal according to the deformation. As an example, the external force detector 310 may include piezoelectric ceramic particles, such as barium titanate (BaTiO₃), or a piezoelectric material, such as PVDF.

The flexible coating part 320 with which the external force detector 310 is coated may be formed by coating the external force detector 310 with a flexible material to prevent the fine protrusion 300 from unintendedly invading a blood vessel or a body cavity and rupturing the corresponding tissue. Also, the flexible coating part 320 may be formed of any one of silicone elastomer, such as polydimethylsiloxane (PDMS) and Ecoflex, and a urethane-based flexible polymer.

According to another exemplary embodiment not shown in the drawings, a fine protrusion 300 may be formed of a composite material in which piezoelectric ceramic particles, such as barium titanate (BaTiO₃), or a piezoelectric material, such as PVDF, for forming an external force detector and a silicone elastomer, such as PDMS or Ecoflex, or a urethane-based flexible polymer for forming a flexible coating part are mixed.

FIG. 7A is a diagram illustrating an example of the apparatus for acquiring biological information according to the exemplary embodiment that is inserted into the aorta V, which is a cardiovascular blood vessel, and detects the rigidity of the aorta, and FIG. 7B is a diagram illustrating a detailed structure of a fine protrusion. In FIG. 7A, fine protrusions 300 come into contact with the inner wall of the aorta V and are deformed. Referring to FIG. 7A, when the apparatus 10 for acquiring biological information is present in a target region, a fluid is introduced through the guide tube 110 and inflates the balloon 100. When the balloon 100 expands, the fine protrusions 300 come into contact with the inner wall of the blood vessel and are deformed.

The external force detector 310 and the flexible coating part 320 output an electrical signal corresponding to the provided external force. The processing unit 400 may receive the electrical signal provided by the fine protrusions 300 and detect the rigidity of the blood vessel.

As an example, when the blood vessel is flexible, the balloon 100 may expand and come into contact with the fine protrusions 300. In this case, not only the fine protrusions 300 but also the blood vessel V is deformed.

However, as elastin providing elasticity to blood vessels disappears due to arteriosclerosis, aging, etc., the blood vessels harden accordingly. Therefore, when the balloon 100 expands and the fine protrusions 300 come into contact with the inner wall of the blood vessel V, the fine protrusions 300 are greatly deformed compared to the blood vessel V. Compared to the former case, a greater external force is applied to the fine protrusions 300, and a greater electrical signal is output by the fine protrusions 300. The processing unit 400 may detect the volume of the fluid introduced through guide tube 110 and the electrical signal output from the fine protrusions 300 and determine the rigidity of the blood vessel V.

FIGS. 6 and 7 illustrates that two electrodes are disposed on the surface of the balloon 100. However, this is merely an exemplary embodiment, and two or more electrodes may be disposed on the surface of the balloon 100. Further, a plurality of fine protrusions 300 may be disposed in one line or an array on one electrode. With such an arrangement structure, it is possible to measure rigidity with high sensitivity and accuracy.

According to the related art, blood flow sensors are attached to the limbs of the human body, and it is determined whether there is a problem in blood vessels by detecting a blood flow rate at a certain location. However, even with the related art, it is difficult to accurately determine which blood vessel is problematic.

According to the exemplary embodiment, it is possible to insert the apparatus 10 for acquiring biological information into a blood vessel and detect the rigidity and the like of the blood vessel from the apparatus 10 in a non-invasive manner.

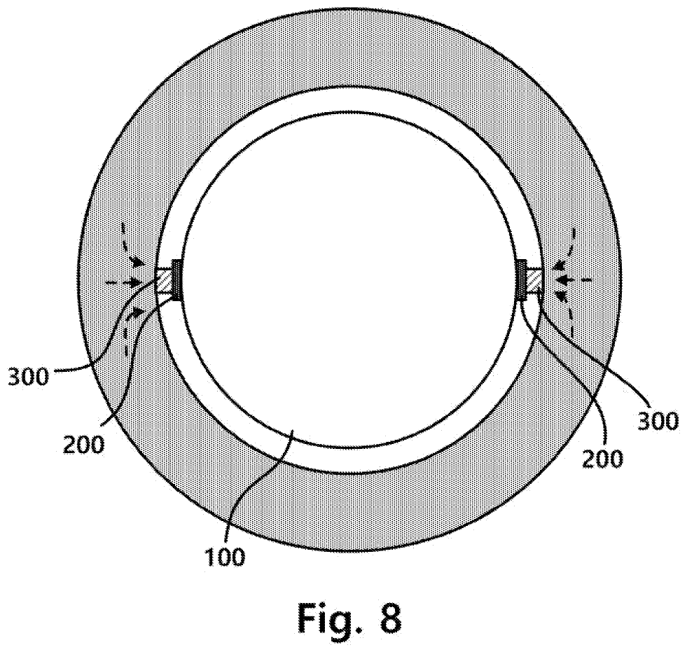

FIG. 8 is a diagram schematically illustrating an operation of still another exemplary embodiment. Referring to FIG. 8, according to the exemplary embodiment shown in the drawing, an apparatus 10 for acquiring biological information is inserted into a body cavity of a blood vessel of a living body. Fine protrusions 300 may be formed of a swelling material, for example, a hydrogel, such as hyaluronic acid (HA) and alginate, or a photocurable hydrogel, such as methacrylate HA (MeHA) or gelatin methacryloyl (GelMA).

In the exemplary embodiment illustrated in FIG. 8, the fine protrusions 300 formed of the swelling material are disposed on electrodes 200. According to another exemplary embodiment not shown in the drawings, electrodes may be formed of a swelling material.

The fine protrusions 300 formed of the swelling material absorb materials at the locations thereof and expand. As an example, in a region in which the apparatus 10 for acquiring biological information is present, the fine protrusions 300 can acquire tissue, such as surrounding tissue, proteins, and body fluids. In addition, immediately after biological information is acquired using electrodes or fine protrusions formed of a swelling material, the biological information can be analyzed. Also, it is possible to widely acquire biological information of a region in which the apparatus 10 for acquiring biological information is present.

Experimental Results

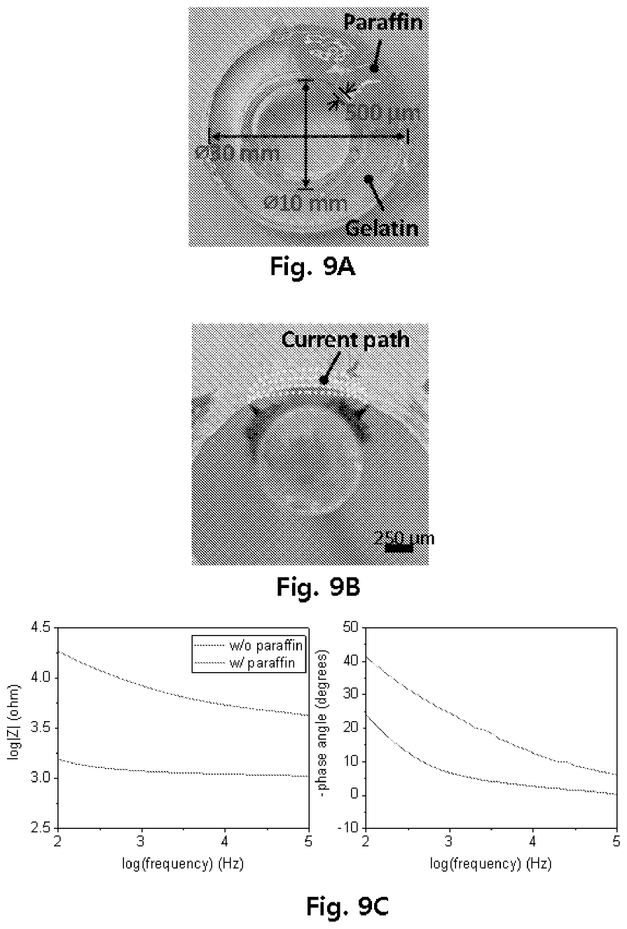

FIG. 9A is an example of simulating a blood vessel with arteriosclerosis. As shown in FIG. 9A, a fibrous capsule of the inner wall of a blood vessel and the blood vessel are simulated with a gelatin layer and paraffin, respectively. The blood vessel phantom was designed with an inner diameter of 10 mm and an outer diameter of 30 mm, and the gelatin layer was designed with a thickness of 500 μm. FIG. 9B is an example of the path of a current when an electrical signal is provided using the apparatus 10 for acquiring biological information according to the exemplary embodiment, and FIG. 9C is a set of graphs illustrating impedance detection results of the above experiment.

Referring to FIG. 9C, the apparatus 10 for acquiring biological information according to the exemplary embodiment can detect an impedance change and a change in impedance phase angle according to a frequency when there is a paraffin layer or not.

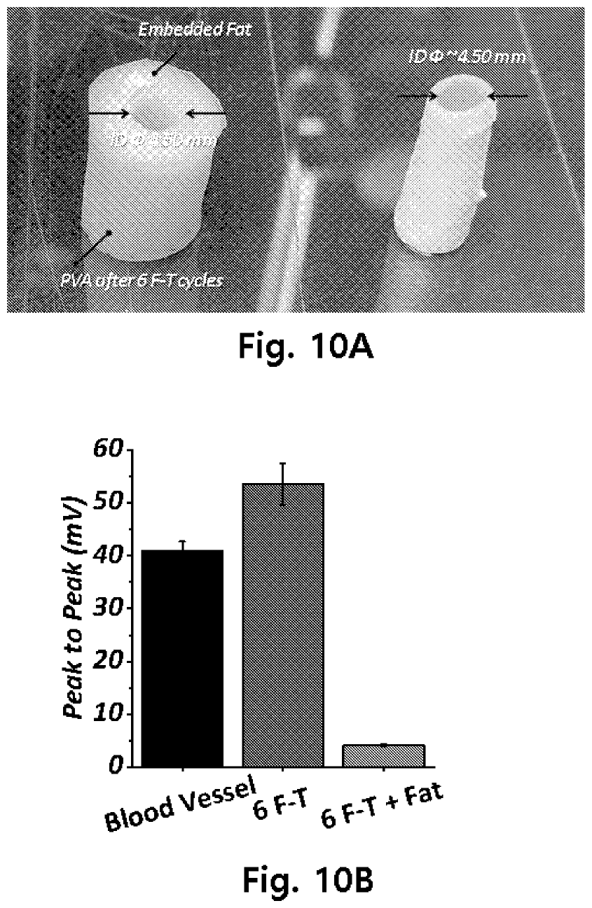

FIG. 10A is a set of views of a blood vessel phantom, which is obtained by freezing and thawing 10% polyvinyl alcohol (PVA) six times to increase stiffness and putting 200 μm of animal fat on the inner wall, and the aorta of a pig.

FIG. 10B a graph illustrating the rigidity of a blood vessel measured with the apparatus 10 for acquiring biological information according to the exemplary embodiment. As shown in the graph, the blood vessel phantom, which is obtained by freezing and then thawing 10% PVA six times to increase stiffness, shows the highest peak-to-peak voltage difference of 55 mV, which indicates the highest stiffness. Next, the blood vessel of the pig shows a peak-to-peak voltage difference of 40 mV. Also, a lipid put in the blood vessel phantom is modeled after vulnerable plaque and shows a peak-to-peak voltage difference of about 5 mV. According to the exemplary embodiment illustrated in the drawing, from the illustrated result, vulnerable plaque can be detected.

Although the present invention has been described above with reference to the exemplary embodiments shown in the drawings, the embodiments are disclosed for implementation and are merely exemplary, and those of ordinary skill in the art should understand that various modifications and equivalents can be made from the embodiments. Therefore, the technical scope of the present invention should be determined by the following claims.

### DESCRIPTION OF SIGNS

10: apparatus for acquiring biological information
20: apparatus for processing biological information
100: balloon
110: guide tube
200: electrode
300: fine protrusion
310: external force detector
320: flexible coating part

What is claimed is:

1. An apparatus for being inserted into a living body and acquiring biological information, the apparatus comprising:
a balloon inserted into the living body to expand or contract; and
one or more electrodes disposed on a surface of the balloon.

2. The apparatus of claim 1, further comprising one or more fine protrusions disposed on surfaces of the electrodes to come into contact with the living body.

3. The apparatus of claim 1, wherein the apparatus is inserted into any one or more of a blood vessel and a body cavity of the living body to acquire the biological information.

4. The apparatus of claim 2, wherein the electrodes include a working electrode and a counter electrode, and
the working electrode and the counter electrode detect any one or more of a current and a voltage generated by oxidation and reduction reactions which occur in the living body coming into contact with the apparatus.

5. The apparatus of claim 4, wherein the fine protrusions disposed on the working electrode and the counter electrode invade the living body such that the oxidation and reduction reactions occur.

6. The apparatus of claim 2, wherein the fine protrusions include a material which provides an electrical signal corresponding to deformation caused by an external force.

7. The apparatus of claim 6, wherein the fine protrusions are coated with a flexible material.

8. The apparatus of claim 6, wherein the fine protrusions include a mixture of a flexible material and the material which provides an electrical signal corresponding to deformation caused by an external force.

9. The apparatus of claim 4, wherein the fine protrusions have a form of a pyramid or a cone.

10. The apparatus of claim 2, wherein the electrodes include a first electrode and a second electrode to which an electrical signal is applied and which detect an electrical signal obtained by providing the electrical signal to the living body, and
the living body and the fine protrusions formed on the first electrode and the second electrode form a path for the electrical signals.

11. The apparatus of claim 10, wherein the electrical signal applied to the first electrode is any one of a voltage signal and a current signal, and
the electrical signal detected by the second electrode is any one of the voltage signal and the current signal.

12. The apparatus of claim 10, wherein the fine protrusions have any one form of a cylinder, a prism, a truncated cone, and a prismatoid.

13. The apparatus of claim 10, wherein the fine protrusions include a conductive material.

14. The apparatus of claim 2, wherein the fine protrusions include a swelling material which absorbs a biological substance at a location where the apparatus is present.

15. The apparatus of claim 1, wherein the electrodes include a swelling material which absorbs a biological substance at a location where the apparatus is present.

16. The apparatus of claim 1, wherein the electrodes are disposed in one or more pairs spaced apart from each other on a surface circumference of the balloon.

17. The apparatus of claim 1, further comprising:
a guide tube through which a fluid flows in or out to expand or contract the balloon; and
a wire connected to the electrodes.

18. An apparatus for processing biological information, the apparatus comprising:
a biological information acquisition part including a balloon inserted into a living body to expand or contract and one or more electrodes disposed on a surface of the balloon; and
a processing unit configured to process biological information acquired by the biological information acquisition part.

19. The apparatus of claim 18, wherein the biological information acquisition part further includes one or more fine protrusions disposed on surfaces of the electrodes to come into contact with the living body.

20. The apparatus of claim 18, wherein the biological information acquisition part is inserted into any one or more of a blood vessel and a body cavity of the living body to acquire the biological information.

21. The apparatus of claim 19, wherein the fine protrusions have a form of a pyramid or a cone,
the electrodes include a working electrode and a counter electrode, and
the processing unit detects any one or more of a current and a voltage generated by oxidation and reduction reactions, which occur in the living body coming into contact with the biological information acquisition part, through the working electrode and the counter electrode and detects whether a lipid, a polyp, or cancer is present at a location where the biological information acquisition part is present.

22. The apparatus of claim 19, wherein the fine protrusions include a material which provides an electrical signal corresponding to deformation caused by an external force, and
the processing unit detects deformation of the fine protrusions caused by an external force as the balloon expands and determines tissue rigidity at a location where the biological information acquisition part is present.

23. The apparatus of claim 19, wherein the fine protrusions include a mixture of a flexible material and a material which provides an electrical signal corresponding to deformation caused by an external force, and
the processing unit detects deformation of the fine protrusions caused by an external force as the balloon expands and determines tissue rigidity at a location where the biological information acquisition part is present.

24. The apparatus of claim 19, wherein the fine protrusions have any one form of a cylinder, a prism, a truncated cone, and a prismatoid,
the electrodes include a first electrode and a second electrode to which an electrical signal is applied from the processing unit and which detect and provide an electrical signal obtained by providing the electrical signal to the living body to the processing unit, and
the living body and the fine protrusions formed on the first electrode and the second electrode form a path for the electrical signals.

25. The apparatus of claim 24, wherein the processing unit provides the electrical signal applied to the first electrode and the second electrode, receives the electrical signal detected by the first electrode and the second electrode, and calculates an electrical impedance of the path.

26. The apparatus of claim 19, wherein the fine protrusions include a swelling material which absorbs a biological substance at a location where the biological information acquisition part is present, and
the processing unit analyzes the biological substance absorbed by the fine protrusions.

27. The apparatus of claim 18, wherein the electrodes include a swelling material which absorbs a biological substance at a location where the biological information acquisition part is present.

28. The apparatus of claim 6, wherein the fine protrusions have a form of a pyramid or a cone.
